(19) United States
(12) Patent Application Publication
(10) Pub. No.: US 2019/0180868 A1
(43) Pub. Date: Jun. 13, 2019

(54) OPTIMIZING EMERGENCY DEPARTMENT RESOURCE MANAGEMENT

(71) Applicant: UBQ. Inc., Clarksburg, CA (US)

(72) Inventors: Maurice Nabil Makram, Sacramento, CA (US); Jamil Hatim Bitar, Sacramento, CA (US); Sarah Jean-Kitazono Heringer, Davis, CA (US); Vinh Quang Le, Roseville, CA (US)

(73) Assignee: UBQ, Inc., Clarksburg, CA (US)

(21) Appl. No.: 16/216,806

(22) Filed: Dec. 11, 2018

Related U.S. Application Data
(60) Provisional application No. 62/597,011, filed on Dec. 11, 2017.

(51) Int. Cl. G16H 40/20; G06Q 10/06; G06N 5/04; G06N 20/00; G16H 50/30; G16H 10/60

(52) U.S. Cl. CPC G16H 40/20; G06Q 10/0631; G16H 10/60; G06N 20/00; G16H 50/30; G06N 5/04

(57) ABSTRACT

The disclosed embodiments disclose techniques for optimizing emergency department resource management. During operation, a system receives a set of parameters that is associated with a patient entering an emergency department. The system analyzes the set of parameters in a machine learning module to determine (1) a calculated acuity score that indicates an estimated severity of illness for the patient and (2) a set of workload predictions that predict a set of resources that will be needed to treat the patient in the emergency department. The system then uses the acuity score and the workload predictions to assign a set of predicted tasks that are associated with treating the patient into the work queues of the emergency department.

### RELATED APPLICATION

This application claims the benefit of U.S. Provisional Patent Application No. 62/597,011, by inventors Maurice Makram, Jamil Bitar, Sarah Heringer, and Vinh Le, entitled “Resource Matching and Sorting of Emergency Department Patients Using Predictive Analytics and Reiterative Learning Algorithms,” filed 11 Dec. 2017, which is incorporated herein by reference.

### BACKGROUND

### Field of the Invention

This disclosure generally relates to techniques for improving the management of hospital emergency departments. More specifically, this disclosure relates to techniques for analyzing and predicting patient issues to optimize resource usage in a hospital environment.

### RELATED ART

Hospital emergency departments are medical treatment facilities that specialize in providing care to patients with acute issues who arrive without prior appointment. Providing emergency care is a particularly difficult problem because such departments need to be able to address a wide, unanticipated range of illnesses and injuries with varying severities in real-time. Emergency departments leverage a substantial set of medical equipment and support staff to assess and treat (or, if needed, escalate) any patient issue that arises. However, managing such infrastructure is complex, and the unplanned nature of patient attendance and needs can lead to the inefficient management of medical resources and patient flows. Studies indicate that the roughly 150 million emergency visits per year in the United States cost some $300 billion, with approximately $40 billion being lost due to inefficient management of patient flows. As an increasing number of people go to emergency departments for unscheduled care, this problem is only likely to get worse.

Improving emergency department efficiency is challenging. One challenge involves sorting and filtering incoming patients. Triage nurses attempt to assess patients to determine an ESI (emergency severity index), but the quality of such assessments may vary greatly in effectiveness across different individuals. Furthermore, because emergency departments leverage a complex network of highly-specialized nurses, doctors, on-call experts, and testing services, managing the flow of patients through the assessment and treatment process can be complex and involve substantial delays. Another issue is that it is very difficult (or even impossible) to predict the number and type of issues that will need to be addressed on a given day, which can lead to a choice between either over-provisioning resources or being caught in a reactive mode with insufficient resources and being overwhelmed.

Hence, what is needed are techniques for managing emergency departments without the above-described problems of existing techniques.

### SUMMARY

The disclosed embodiments disclose techniques for optimizing emergency department resource management. During operation, a system receives a set of parameters that is associated with a patient entering an emergency department. The system analyzes the set of parameters in a machine learning module to determine (1) a calculated acuity score that indicates an estimated severity of illness for the patient and (2) a set of workload predictions that predict a set of resources that will be needed to treat the patient in the emergency department. The system then uses the acuity score and the workload predictions to assign a set of predicted tasks that are associated with treating the patient into the work queues of the emergency department.

In some embodiments, the patient parameters include:
a set of patient vital information that is measured at the time the patient checks in to the emergency department. These “vitals” may include the patient's heart rate, blood oxygen levels, and blood pressure.
patient information recorded by a triage nurse that includes the patient's age and chief complaint.
identifying information that is used to access the patient's medical history via the patients electronic health record; information from the patient's electronic health record is included in the parameters that are analyzed by the learning machine module.

In some embodiments, the patient vitals are measured in real-time using a hardware-based palm reader that also reads patient biometric information that is compared to the patient's identifying information to authenticate the patient and facilitate access to the patient's electronic health record.

In some embodiments, the triage nurse also records for the patient an intuited numerical acuity score that is based on the nurse's experience, training, and gestalt. The machine learning module analyzes and compares the calculated acuity score and the intuited acuity score to check that the triage nurse and the machine learning module approximately agree in the assessment of the patient and are not missing any illness factors. If the calculated acuity score and the intuited acuity score diverge substantially, the system may flag a warning and allocate additional resources to determine an accurate acuity level for the patient.

In some embodiments, the machine learning module tracks (1) a set of patient and emergency department input parameters for patients who visited the emergency department previously, (2) the workload that was used to treat each patient, and (3) the set of outcomes for each previous patient. The machine learning module uses supervised machine learning techniques to match the set of parameters for the current patient to a statistically matching set of tracked input parameters to determine workload predictions and a predicted outcome for the patient.

In some embodiments, the tracked parameters that are tracked and used by the machine learning module for supervised machine learning comprise: an emergency department census at the time of the patient's assignment; an inpatient census at time of the patient's assignment; the patient's ambulatory status; the means with which the patient arrived; the patient's triage vital signs and timestamp; the patient's blood pressure; the patient's heart rate; the patient's respiratory rate; the patient's temperature and method of measurement; the patient's weight; the patient's pulse oxygen saturation; the patient's problem list and medical history; the patient's current medications; the patient's allergies; the patient's social history; the patient's registration timestamp; an emergency department provider to which the patient was assigned and a timestamp for the provider assignment; an emergency department nurse to which the patient was assigned and a timestamp for the nurse assignment; an emergency department room to which the patient was assigned and a timestamp for the room assignment; a set of studies ordered for the patient, along with the timestamps of when the studies were ordered, when the studies had changes, and when the studies were completed; a disposition decision for the patient and a timestamp for the disposition decision; an assignment for an inpatient bed for the patient and a timestamp for the assignment of the inpatient bed; a final disposition for the patient and a timestamp for the final disposition; a billing level of service associated with the patient; and current environmental conditions for geographic vicinity of the emergency department for the timeframe in which the patient entered the emergency department.

In some embodiments, the calculated acuity score indicates an estimated severity of illness for the patient and is used to sort the patient into one of a set of four acuity groupings that comprise (1) resuscitation, (2) high acuity, (3) medium acuity, and (4) low acuity.

In some embodiments, the workload predictions determined by the machine learning module include a set of medications that will need to be administered to the patient, a set of laboratory orders that will need to be applied for the patient, a set of radiology orders that will need to taken for the patient, an amount of time that a physician will need to spend with the patient; and a set of procedures that the patient will likely need to undergo.

In some embodiments, the process of assigning the predicted tasks considers the shift times for the staff currently working in the emergency department, current and predicted patient arrival patterns for the emergency department, and the acuity scores and workload predictions for all of the patients who are currently active in the queues of the emergency department.

In some embodiments, assigning the set of predicted tasks further comprises prioritizing the flow of patients through the radiology, laboratory, and discharge queues.

In some embodiments, the patient is immediately assigned to a specific physician at the time that the set of predicted tasks are assigned into the work queues of the emergency department. Immediately assigning the patient to the specific physician provides accountability that leads to better care and efficiency for tasks associated with the patient.

In some embodiments, assigning the predicted tasks into the work queues further comprises sending push notifications to the set of staff and resources associated with a treatment plan for the patient to notify them of their new assignments for the patient.

In some embodiments, a crowding score for the emergency department is determined based on the number of patients currently being treated by the emergency department; the acuity scores of the patients currently being treated by the emergency department; the number of patients currently arriving at the emergency department; the set of resources available in the emergency department; the status of the radiology, laboratory, and discharge queues for the emergency department; and the number of hospital patients boarded in the emergency department.

In some embodiments, the crowding score is used to calculate a crowding coefficient for the emergency department. The set of active resources of the emergency department can be adjusted based on this crowding coefficient. For instance, additional staff resources may be activated in real-time to reduce a bottleneck, and/or one or more staff resources that are currently underused (and predicted to not be used in a proximate timeframe) maybe be deactivated or shifted to a different care area in real-time to reduce costs and/or improve efficiency in a different care area.

In some embodiments, determining a physician assignment for the patient is based on (1) the patient's calculated acuity score, (2) a workload prediction for the patient that is associated with physician time, effort, and experience, (3) the physician's shift time, (4) the length of the physician's shift, (5) the crowding coefficient; and (6) the number of patients being treated (both collectively and individually) by the physicians who are currently active in the emergency department.

In some embodiments, the tracking system may determine that a task associated with the patient is its predicted workload. In response, the system may rebalance one or more queues that are affected by the excessive task in real-time using the acuity scores and workload predictions of all of the other patients in the emergency department to adjust for the excessive task. After the patient's treatment has completed, the machine learning module is updated with the actual workloads that were performed for the patient so that the patient's workloads can be used to train the machine learning module in future predictions.

### BRIEF DESCRIPTION OF THE FIGURES

FIG. 1 illustrates an exemplary system in which the flow of emergency department operations is split across four phases in accordance with an embodiment.

FIG. 2 illustrates an exemplary biometric assessment device in accordance with an embodiment.

FIG. 3A illustrates an exemplary scenario in which a queue of incoming patients are assessed and assigned to a set of emergency department queues in accordance with an embodiment.

FIG. 3B illustrates a set of emergency department queues in the context of the scenario illustrated in FIG. 3A in accordance with an embodiment.

FIG. 4 illustrates a computing environment in accordance with an embodiment.

FIG. 5 illustrates a computing device in accordance with an embodiment.

FIG. 6 presents a flow chart that illustrates the process of optimizing emergency department resource management in accordance with an embodiment.

FIG. 7A illustrate a set of exemplary collected patient data in accordance with an embodiment.

FIG. 7B illustrate a set of exemplary data visualization for collected patient data in accordance with an embodiment.

### DETAILED DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown, but is to be accorded the widest scope consistent with the principles and features disclosed herein.

The data structures and code that execute techniques described in this detailed description are typically stored on a non-transitory computer-readable storage medium, which may be any device or non-transitory medium that can store code and/or data for use by a computer system. The non-transitory computer-readable storage medium includes, but is not limited to, volatile memory, non-volatile memory, magnetic and optical storage devices such as disk drives, magnetic tape, CDs (compact discs), DVDs (digital versatile discs or digital video discs), or other media capable of storing code and/or data now known or later developed.

The methods and processes described in the detailed description section can be embodied as code and/or data, which can be stored in a non-transitory computer-readable storage medium as described above. When a computer system reads and executes the code and/or data stored on the non-transitory computer-readable storage medium, the computer system performs the methods and processes embodied as data structures and code and stored within the non-transitory computer-readable storage medium.

Furthermore, the methods and processes described below can be included in hardware modules. For example, the hardware modules can include, but are not limited to, application-specific integrated circuit (ASIC) chips, a full-custom implementation as part of an integrated circuit (or another type of hardware implementation on an integrated circuit), field-programmable gate arrays (FPGAs), a dedicated or shared processor that executes a particular software module or a piece of code at a particular time, and/or other programmable-logic devices now known or later developed. When the hardware modules are activated, the hardware modules perform the methods and processes included within the hardware modules.

### Limitations of Existing Emergency Department Practices

As mentioned above, studies indicate that there are presently roughly 150 million emergency visits per year in the United States. At an average cost of $2000 per visit, the revenue from these visits is a considerable $300 billion. It is estimated that approximately $40 billion of this amount is lost every year in emergency departments (EDs) due to the inefficient management of resources and patient flows. Another factor is the number of people who leave without being seen (“LWBS”), which is estimated to have averaged 2%. Those additional 300,000 aborted visits represent another approximately $0.5 billion in lost revenue. In addition to financial concerns, inefficiency and related stress are also a major negative factor. Patients left waiting due to inefficient patient flows suffer and experience a negative patient experience. Physicians make individual heroic efforts to provide the best care possible, but the limitations of an inefficient environment can result in low quality of care and compassion fatigue.

Consider the patient input process for a typical emergency department. Triage nurses assess patients to determine an ESI (emergency severity index), which is a five-level resource prediction tool that is used to triage and sort patients based on the urgency of their condition and also serves as the basis for staffing models in many emergency departments. One issue with the ESI is that it relies on human intuition, hunches, training, and experience, which can vary widely across individual providers, and thus unfortunately can lead to variability that can affect the timing, reliability, and validity of the assessment. One provider may be able to provide an accurate assessment for a given condition in minutes, while another may take much longer and have substantially lower accuracy. The quality, efficiency, and overall experience of care depends very heavily on this initial assessment step.

Another factor in emergency department inefficiency is the complex network of resources that need to be managed. The need to be able handle a wide (and changing) range of patient conditions involves maintaining a complex network that includes highly-specialized emergency department nurses and doctors, on-call experts (e.g., in surgery, obstetrics, general medicine, cardiac care, etc.), and networks of testing services (laboratories, radiology, imaging, etc.). Managing availability, queuing, dependencies, interactions, and outputs between all of these entities is complex, and typically involves a number of queues that are difficult to optimize without a clear high-level view of all of the patients that are currently being treated and the specific resources that are currently available (and their status).

Another potential issue can be the misalignment of provider and patient interests. The traditional ED model seeks to identify the patients who are the most ill, with the other patients left waiting until their lower-severity issues can be addressed. While this model does allow the most serious cases to be handled first, serious illnesses are not always readily apparent to other waiting patients, and this can foment resentment and frustration.

In some cases physician agendas may also not align completely with optimal efficiencies. For instance, an exhausted physician near the end of their shift may attempt to perform “chart picking” to choose patients with more manageable issues that can be addressed more quickly, or physicians may seek to avoid seeing patients against which they have particular biases. Such local adjustment attempts can lead to sub-optimal patient flows and delays. Without central oversight it is difficult to prevent such issues, but such oversight often injects additional complexity and delay in the entire assessment and treatment process. A typical patient does not have an advocate or any control in this process.

Emergency departments remain the place most people go for unscheduled care, and hence existing problems will only continue to increase as the number of visits grows. The techniques described in the following section disclose techniques for improving the flow of patients and the management of ED resources to remove barriers, minimize waste, and provide the best possible care for every patient and every ED visit.

### Improving Emergency Department Efficiency

In some embodiments, a predictive assessment and assignment system overcomes the above-described ED issues by using deep-learning techniques that track and analyze a range of patient and ED factors to predict and schedule the resources that will be needed to care for arriving emergency department patients. Data science and queuing-theory techniques are applied to patient data to calculate an assessment score for each incoming patient, and this assessment score is then used in conjunction with the tracked current status of the ED to determine a proposed flow of action(s) for that patient. The prediction techniques track patient and process (e.g., resource) outcomes to make adjustments as needed and to improve resource matching accuracy over time via reiterative learning techniques.

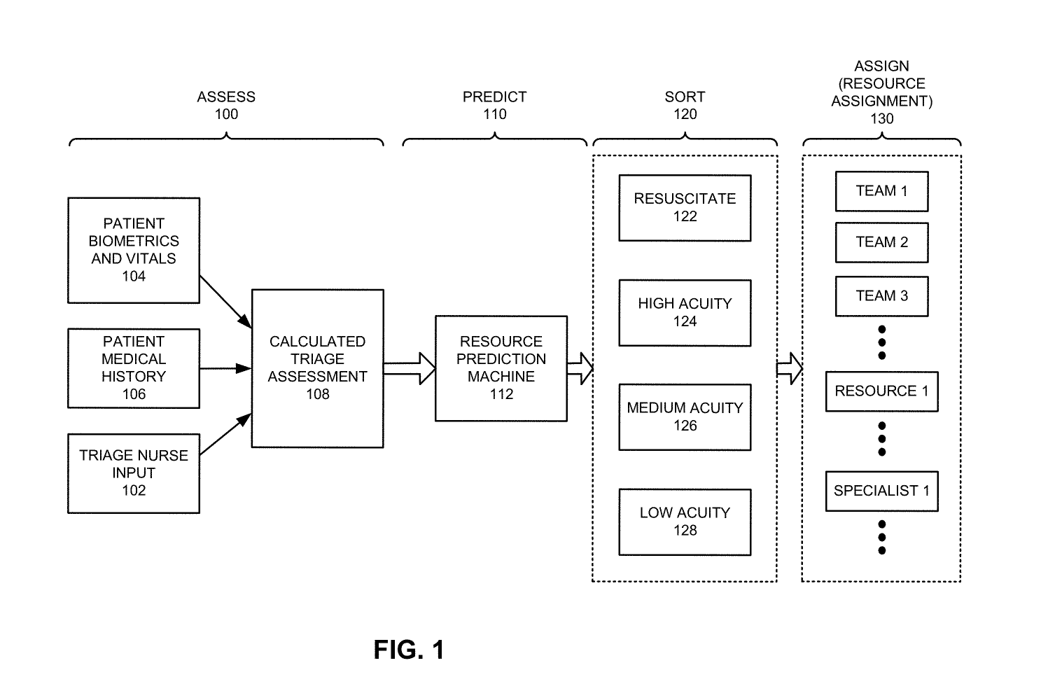

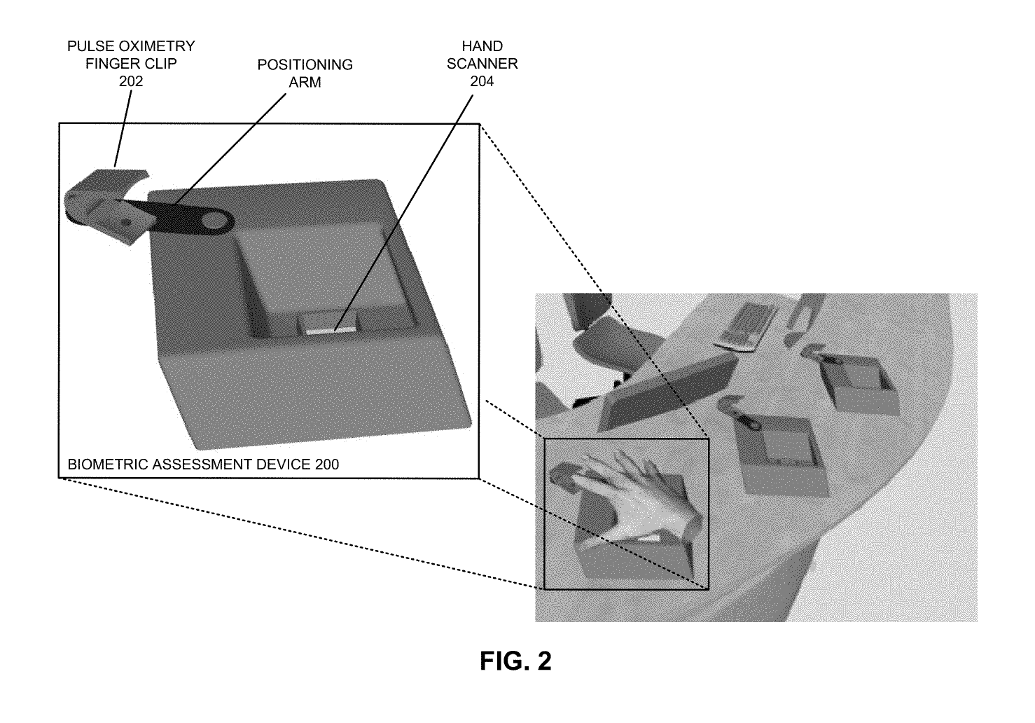

FIG. 1 illustrates an exemplary embodiment of the disclosed system in which the flow of ED operations is split across four phases, specifically the assess 100, predict 110, sort 120, and assign 130 phases. In the assess phase 100, a patient entering the emergency department is first assessed 100 using a set of data sensors that gather an initial set of patient biometric and sensor data 104. For instance, when first checking in a patient may be scanned using one or more networked devices that gather biometric and status data. For example, a biometric assessment device 200 (as illustrated in FIG. 2) that includes a pulse oximetry finger clip 200 and a hand scanner 204 may be used in conjunction with a blood pressure sensor to quickly gather an initial set of patient vital information (“vitals”) that comprise heart rate, blood oxygen levels, blood pressure, etc. In parallel, a triage nurse enters into the system a set of input 102 that includes the patient's chief complaint, age, and a link to the patient's medical history 106. In some embodiments, the patient biometrics gathered by the biometric assessment device 200 may also be compared with information in the patient's medical history 106 (e.g., via the patient's electronic health record, or EHR) to authenticate that the patient has been identified correctly.

The disclosed system uses the received inputs 102-104 to calculate a triage assessment 108 for the patient, which is then input into a resource prediction machine 112 that leverages supervised machine learning techniques to predict a set of workloads and resources that will be needed for the patient based on previously-tracked patients with similar parameters. The calculated triage assessment 108 and predictions are also used to help determine a predicted severity for the incoming patient (e.g., whether the patient needs to be resuscitated 122 or, if responsive, is suffering from an issue of high 124, medium 126, or low 128 acuity). The assessment 108 and predictions are then used to assign the patient to a specific care team and allocate the resources that are predicted will be needed to care for the patient. Note that numerical acuity assessments are typically directly correlated with at least one of the severity of the patient's issue and the predicted amount of resources that will be needed to treat the patient.

In some embodiments, a calculated triage assessment (also referred to as an “iAcuity score”) replaces, or can be used in conjunction with, a traditional numerical ESI score that is determined by a triage nurse (e.g., based in part on the triage nurse's experience and intuition). For instance, in some emergency departments, an ESI of 4-5 might be associated with low acuity, an ESI of 3 might be associated with medium acuity, and an ESI of 1-2 might be associated with high acuity. Utilizing multiple data inputs that include data points such as that patient's age, chief complaint, vitals, and past medical history facilitates sorting patients into levels of acuity accurately and consistently. While in the long term a learned, statistical value learned from a large sample of historical patients will provide a more deterministic and reliable assessment, comparing the two values can serve as a check and confirmation, with a disparity in the two assessments serving to give feedback to the other mechanism that something may have been missed. If the calculated acuity score and the intuited acuity score diverge substantially the system may flag a warning or otherwise escalate the issue in some other way. For instance, options for handling such situations may include one or more of: (1) bringing the disparity to the attention of a physician, (2) temporarily increasing the acuity level for the patient until the disparity can be understood and resolved, and/or (3) assigning the patient to the higher of the two acuity levels (if they are different) until the patient has been evaluated. The level of disparity that merits flagging a warning and/or escalating the situation may vary; in some embodiments a standard range may be configurable, as are the set of actions to pursue depending on how much the values differ.

In some embodiments a wide range of factors are tracked by the disclosed system to facilitate a consistent process that can identify patients with immediate and/or urgent needs and give accurate predictions. For instance, the set of patient and general status data that is tracked may include (but is not limited to):
ED census at the time of the patient's assignment
Inpatient census at time of assignment (inpatient beds, ICU beds)
Ambulatory status
Means of patient arrival (EMS, police, walk-in, etc)
Patient triage vital signs+timestamp
Patient blood pressure
Patient heart rate
Patient respiratory rate
Patient temperature+method of measurement
Patient weight
Patient pulse oxygen saturation
Patient problem list/medical history
Patient medications
Patient allergies
Patient social history (smoker status, alcohol history, drug history)
Registration timestamp
Assigned ED Provider+timestamp
Assigned ED Nurse+timestamp
Assigned ED Room+timestamp
Studies ordered: CT scans, x-rays, MRIs, ultrasounds, labs
Study order time timestamps
Study order status change timestamps
Study order complete timestamp
Disposition decision+timestamp
Assigned inpatient bed+timestamp
Final disposition+timestamp
Time of disposition from: ICU, Telemetry, Med-Surgery, etc. timestamps
Billing Level of Service+Procedures for Nursing and Providers
Current environmental conditions (e.g., temperature, air quality, etc.)
   Tracking, compiling, and analyzing the above factors for each patient over time provides a large set of correlations that can be leveraged for each subsequent patient. For instance, one correlation might be able to generate a accurate assessment score based on an initial triage consultation and the patient's vitals, chief complaint, and past medical history. The system may also track correlations between length of stay in the emergency department and the patient's vitals, chief complaint, and past medical history. The availability of such data enables data mining and learning techniques that guide future care decisions. Environmental effects are also an important point of consideration—for example, patient emergency room arrival patterns may follow historical patterns (e.g., specific times of day or weekend) or be affected by environment factors such as pollen counts, weather, natural disasters, etc. Data gathered over time can identify the impact of such factors and then facilitate modifying how parameters are weighed during subsequent reoccurrences to ensure that staffing and resources are adequate to meet needs. Gathering and analyzing such data can also lead to the discovery of underlying trends that might otherwise not be noticed.

In some embodiments, deep learning techniques leverage previous patient visits and scenarios by performing pattern recognition on historical ED patient visit data to predict likely resources that will be needed (e.g. projected staffing and resource needs) and patient outcomes in the present. More specifically, supervised machine learning techniques that track previous input-output pairs can use the tracked data as training data for accurately predicting/inferring both known and unknown patient scenarios; previous outcomes are measured and used as guides in learning to predict future behavior (e.g., via reiterative learning techniques). For instance, the disclosed techniques can use historical tracked correlations between the above data fields from previous patients and their resulting (historical) workloads to determine in a standardized way a current set of work coefficients (e.g., projected workloads for the illnesses currently being handled) that estimate current workload and predict new patients are likely to affect the current workload. For example, parameters and/or outputs may include the current weighted work load (e.g., current physician, registered nurse, and imagining or laboratory service workloads), the predicted work load of a patient before the patient is assigned (based on weightings derived from other patients with similar symptoms and characteristics noted upon entry to the emergency room), and recourse predictions (e.g., the likelihood based on the statistical tracked histories of other patients that a given patient will be either discharged, admitted to the EM, or admitted to an intensive care unit (ICU)). Leveraging the tracked historical visit data and their outcomes enables the disclosed learning platform to bring unprecedented precision to ED resource prediction and flow.

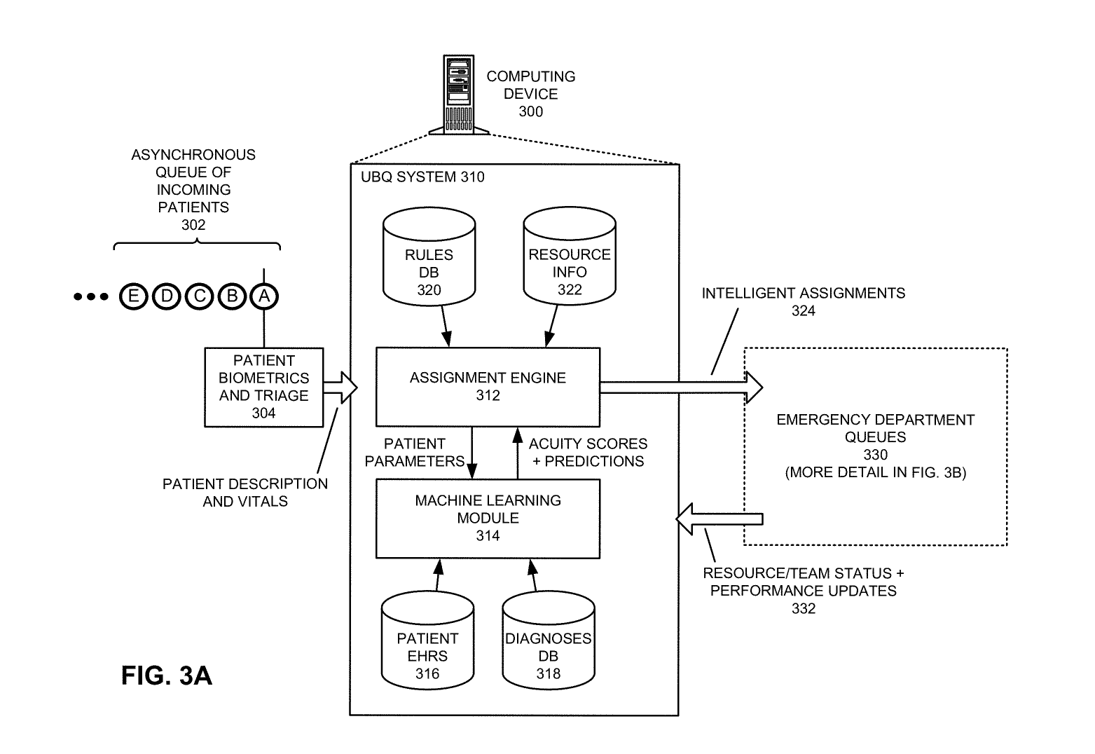

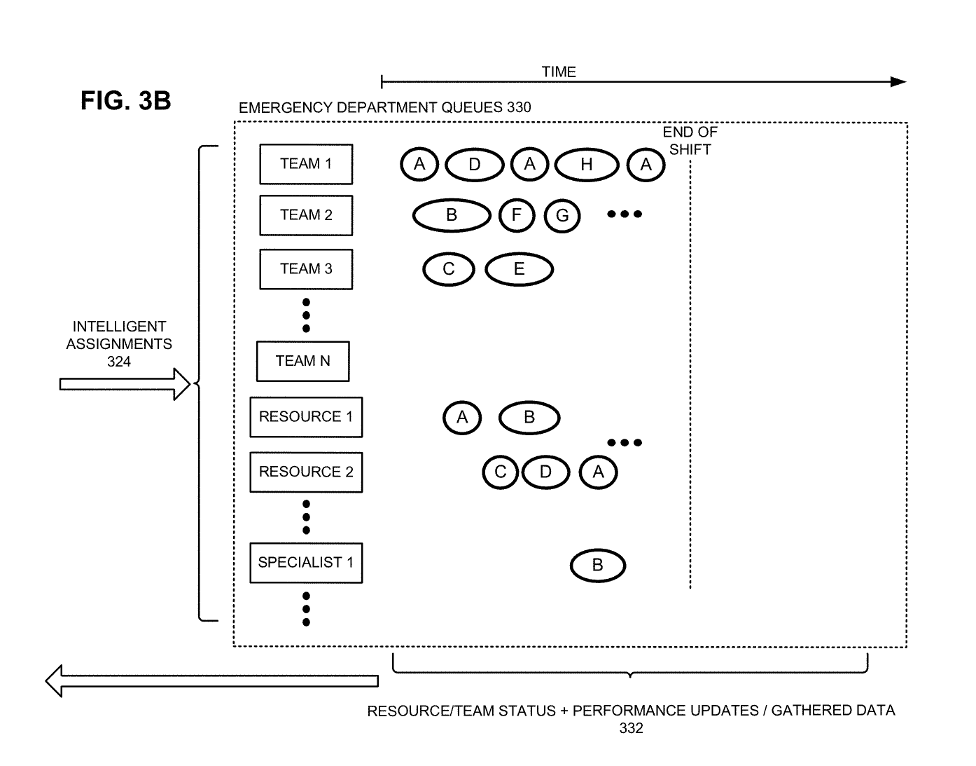

FIGS. 3A-3B illustrate another, more detailed, embodiment of the disclosed system in operation, executing on a computing device 300. Note that while FIG. 3A illustrates components of the disclosed system executing on a single computing device 300, the disclosed techniques are not limited to such a configuration, and would benefit from executing on a larger computing platform. For instance, the illustrated data repositories, assignment engine, and machine learning module can be executed in a data center and/or a scalable cloud computing environment that can rapidly process the constantly-growing tracked historical data set to generate predictions and workloads. Such infrastructure and tracking efforts can also be shared across multiple sites and emergency departments; collecting a larger data set further improves the accuracy of results and facilitates sampling across a wide range of locations and conditions. Furthermore, such infrastructure may also be replicated in additional remote and local installations to ensure that life-critical prediction and assignment services are always available in case of failures. A multi-site architecture also facilitates bringing new and/or additional EDs into the system—instead of having to build a data set from scratch, a new site can immediately benefit from predictions made by the preceding participating sites. In some embodiments, some aspects of the tracked data may be anonymized to ensure that patient confidentiality is maintained in the shared environment.

In FIG. 3A, an asynchronous queue of incoming patients 302 entering an emergency department are checked in at a biometrics and triage station 304 where they identify themselves, describe their illness, and undergo an initial scan via the above-described data sensors. The resulting patient description and vitals are submitted to the UBQ system 310, which comprises an assignment engine 312 and a machine learning module 314. The patient parameters are forwarded to the machine learning module 314, which can access a database of patient electronic health records (EHRs) 316 to look up the health record of the specified patient to access relevant historical information that can supplement and/or explain the patient's current parameters. In some embodiments, a patient may also be associated with data from another hospital system that is imported by UBQ system 310.

Machine learning module 314 then compares the set of patient parameters to the tracked history of past inputs, outcomes, and diagnoses that have been collected by the system in a diagnoses database 318 to determine an acuity score and workload predictions for the patient. Note that the workload predictions can span a range of resources, for example considering the number of medications the patient takes and/or needs, the set of laboratory and radiology that are likely to be ordered, and the amount of time a physician is expected to need to spend with the patient as well as any potential procedures. These workload predictions are passed to the assignment engine 312, which generates a set of intelligent assignments 324 for the patient that are added to the emergency department queues 330 (illustrated in more detail in FIG. 3B). In some embodiments, the supervised learning techniques leveraged by machine learning module 314 may involve: (1) determining operational performance metrics for the emergency department setting based on information from one or more data sources, (2) determining an expected value for the operational performance metric, (3) associating a set of potential correlational factors for each operational performance metric, and (4) determining the significance of the correlational factors on the deviation of the operational performance metric from its expected value by using supervised machine learning.

Assignment engine 312 receives real-time status updates about outcomes and resource usage in the ED (e.g., the availability, load, and progress of physicians, nurses, specialists, diagnostic equipment, etc.), and considers a number of factors when formulating assignments. Status information is tracked in a resource information database 322 that assignment engine 312 consults when making subsequent assignment decisions. Assignment engine 312 also can be configured with a set of local rules that are specified by an administrator via a rules database 320, which allows individual EDs to tailor the UBQ system 310 to their specific needs. More specifically, the emergency room administration may develop and/or identify a set of rules that substantially increase the productivity and efficiency of emergency department workflows. For instance, such rules may limit the number of patients per hour for a certain segment of care (e.g., determining that low acuity efforts are maximally efficient if no more than six patients per hour are assigned to that category), or specify that physicians at the beginning of a shift only receive a certain severity of patients (e.g., physicians starting a fresh shift in the emergency department start off by being assigned patients with a medium acuity range of an ESI of three). Another exemplary rule leverage a knowledge that the radiology, laboratory, and discharge operations often become bottlenecks, and hence prioritize the flow of patients through the radiology, laboratory, and discharge queues (thereby potentially removing bottlenecks for other workflows in the ED and also releasing some patients from the ED completely and enabling those patients to be removed from further workflow considerations). Note that the learning techniques described for the disclosed system can be used to identify and further tune such rules over time as large sets of patient progress data and correlations are tracked. In some embodiments, data mining techniques can be used to traverse the tracked data to determine correlations between more successful treatment and assignments and develop such rules.

Note that acuity scores and workload predictions may be specified in a number of different representations. In the traditional ED model, an ESI score is a single digit number determined by a triage nurse; a calculated iAcuity score may be a similar single-digit, statistically-calculated score (that could be directly compared to a human-determined ESI value). Alternatively, in some embodiments, an iAcuity score may be a set of multiple values that describe different aspects of a patient's condition. Workload predictions (also referred to as “workload coefficients”) may also be described using a number of different formats. In general, workload predictions attempt to convey a prediction of how much work a patient will generate in the ED, which can encompass both staff time and effort as well as specific resources and/or tests that need to be involved. For instance, workload predictions might be conveyed in predicted units of time, or might be left more generally as units of relative effort that distinguish different operations. For example, treating a toothache may be predicted to generally take half the time of performing stitches for a light wound, while a patient with gastrointestinal bleeding that involves blood transfusions, multiple consultant specialists, and preparing an operating room may be predicted to take orders of magnitudes more time to treat. Regardless of the specific metric that is used, assignment engine 312 is able to compare the workloads of different physicians and resources, compare the incoming assessed patient's workload to the existing queued patients, and make assignments that balance the workload evenly across the human and equipment resources in the ED.

An important aspect of the assignment process is that patients are immediately assigned to a physician provider after their initial assessment. Where the traditional ED model typically has other patients wait until higher-severity patients have been taken care of and a physician becomes available, the disclosed system instead already assigns incoming patients to physicians based on the known set of predicted workloads. More specifically, because the system has a global knowledge of what is happening in the ED and a set of predictions for everything that is currently being handled, it can already decide based on the predictions which physician should be assigned the new patient, and make an assignment. The patient may not immediately see this physician (i.e., that physician may still have other patients ahead of the new patient in their incoming queue), but the act of assigning the patient to the physician already at the time they arrive provides a level of accountability that leads to better care and efficiency. Such information may also already be conveyed to the patient (e.g., “You will be seeing Dr. Smith, who currently has two patients ahead of you on their queue”)—conveying early status updates to patients provides reassurance and a feeling of progress, thereby substantially improving the patient experience. Assignments can also be conveyed to staff using push notifications that communicate queue changes and/or additions.

FIG. 3B illustrates an exemplary set of assignments that are assigned to emergency department queues 330 in response to the incoming patients illustrated in FIG. 3A. In some embodiments, one aspect of intelligently assigning work to different teams involves ensuring that all of the patients with complicated issues are not assigned to the same physician/team; overwhelming one specific provider can lead to switching overhead, exhaustion, and inefficiency. Note that patient issues often involve multiple tests or diagnostics spread out over multiple queues. For instance, in the context of FIGS. 3A-3B, patient A is: (1) first seen by team 1, then (2) diagnosed by resource 1 (e.g., an X-ray service), (3) seen by team 1 again, (4) diagnosed using resource 2 (e.g., an MRI or CT scan), and then (5) seen by team 1 again. These types of patterns are tracked (in association with their related illnesses or injuries), and can already be anticipated predicted by machine learning module 314 based on the patient's input parameters and in turn considered by assignment engine 312 when assigning that patient to queues. Consider a detailed example of an incoming patient that complains a severe issue (e.g., “crushing chest pain”), for whom the initial triage and vitals indicate high acuity, and who has supplementary parameters in the EHR. Machine learning module 314 determines a high acuity from these inputs, and predicts a large set of laboratory work, imaging, and/or consultation/monitoring based on historical situations with similar parameters. These predictions are conveyed to the assignment engine 312, which identifies that this will be a time-consuming patient and ensures that the physician/team being assigned this patient has both the current and future capacity budgeted in to accommodate the needed care for this patient.

In some embodiments, the assignment system strives to predictably divide the load across the resources of the emergency department. An ideal set of assignments have physician teams accepting and releasing patients at a continuous and equal long-term rate; this typically correlates to the best throughput results and also in the best care. More specifically, this indicates that patients have a faster average time to see a physician, and less waiting delay and smooth throughput generally correlate with less waste and higher efficiency. Predictively dividing difficult cases across resources facilitates balanced, smoother flows than allow new patients to be seen in a sooner timeframe than in the traditional model.

In some embodiments, the disclosed system leverages the knowledge of the predicted workloads and acuity scores of current patients and the knowledge of the queues for the emergency department to determine a crowding score for the emergency department. For instance, this value may be based on factors that include, but are not limited to: the number of patients currently being treated, their acuity scores and predicted workloads, the number of new patients currently arriving at the emergency department, the set of resources available, the status of the radiology, laboratory, and discharge queues, and the number of hospital patients that are currently being boarded in the emergency department (e.g., patients that have been admitted to the hospital but need to be boarded in the emergency department for some timeframe, for instance because the hospital does not have room and/or staff available to handle those patients yet). This crowding score can be used to calculate a crowding coefficient that can be used to determine in real-time whether to activate or deactive resources in the emergency department. For instance, the system may indicate that some resources are not being used and are not likely to be used based on the incoming predicted workloads, and also determine other resources that are becoming bottlenecks and should be increased. If needed additional staff resources can be called in as needed, but often existing resources can also be rebalanced to manage surges and/or overload in specific areas. For example, if physicians are specifically assigned to acuity groups and the low-acuity groups are overwhelmed with patients, new low-acuity patients may temporarily be assigned to medium-acuity physicians to manage a temporary surge (or to help manage the surge until other resources can arrive).

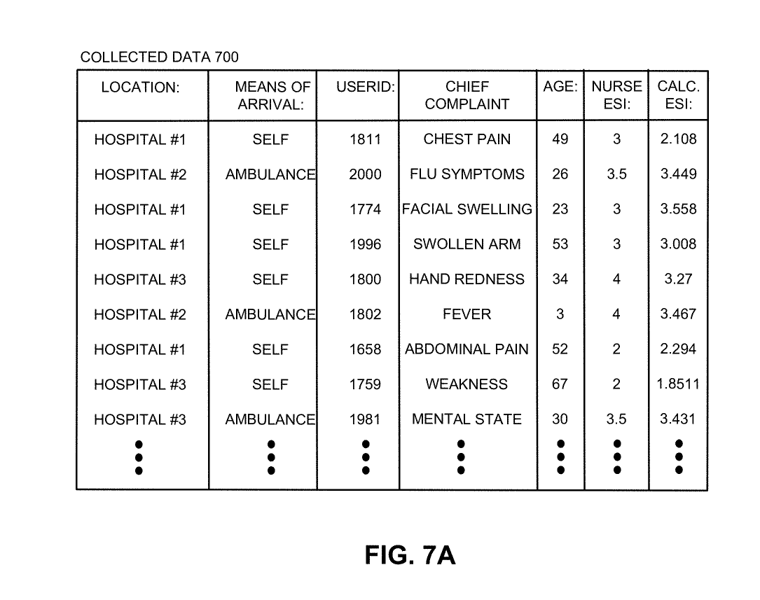

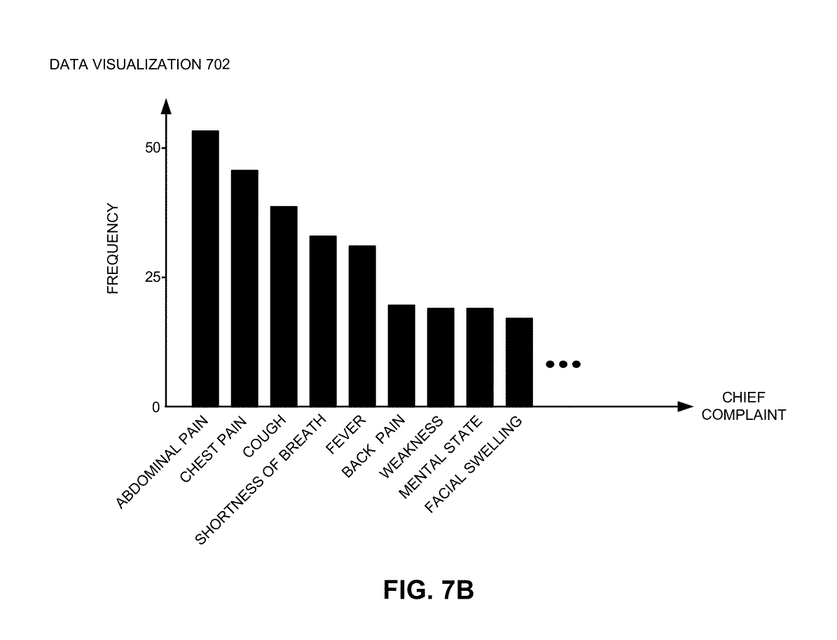

In some embodiments the set of collected data can be viewed and analyzed by both the machine learning system and medical administrators to gain insight into emergency department and patient trends. FIGS. 7A-7B illustrate a set of collected data 700. For instance, FIG. 7A illustrates a chart that lists a recent set of patients that were treated at a number of locations. Factors that are considered and displayed include the means of arrival (e.g., either arriving themselves vs via an ambulance), their chief complaint, age, a triage nurse's ESI assessment, and the calculated ESI. Such information can assist administrators and the machine learning module in determining how close the nurses' and calculated ESI values are. The compiled data can also be organized into visualizations that provide insight into ED operations. For instance, in FIG. 7B data visualization 702 illustrates a histogram of the most common chief complaints encountered in a specific time period.

Note that one aspect of the disclosed learning techniques is that the machine learning module also determines a set of predicted outcomes that are associated with the input patient parameters (in addition to the iAcuity score and the predicted workloads). For instance, outputs may include correlated predicted length of stay in the ED and/or hospital, and a predicted disposition (e.g., whether the patient is likely to be sent home, to the ICU, operating room, inpatient care, etc.). In some embodiments, the assignment system determines that the predicted outcome is severe, and can already initiate a higher level of care. For example, the assignment system may be able to determine ICU occupancy, and already reserve a bed for an incoming patient in the ICU based on the severe predicted outcome. Such capabilities, in combination with the average reduction in time to see a physician and patient flows enabled by the prediction and assignment systems, can make a significant positive impact on patient care outcomes.

Note also that the disclosed learning techniques constantly learn and adapt by incorporating current parameters and outcomes into the tracked set. In the exemplary scenario of FIGS. 3A-3B, resource/team status, performance updates, and gathered outcome data 332 is conveyed back to the UBQ system 310, allowing the system to determine how the actual outcomes compared to the prediction. The new outcomes are added to the training data and incorporated into future predictions and assignments to ensure that the system continues to improve in accuracy over time. This feedback can also be analyzed to identify queue congestion and/or inefficiencies in real-time, and if needed can alert administrators of issues. For example, the system may raise a flag if a patient is determined to be using a much higher level of care than was predicted so that the case can be escalated as needed (e.g., modifying queues in real-time if needed to accommodate) and, if necessary, someone can determine why the reality diverged from the predictions. The tracking capabilities and feedback mechanisms can also be used to identify particular staff expertise that leads to higher-than-expected efficiency, potentially allowing administrators to determine and/or improve best practices. Furthermore, in some embodiments, patients can be enabled to provide feedback to the system as well. The disclosed techniques facilitate conveying more information to patients, giving them a sense of control in their care. Patient input via satisfaction scores can be used to determine whether the system is working well from the patient perspective, and potentially make adjustments if needed.

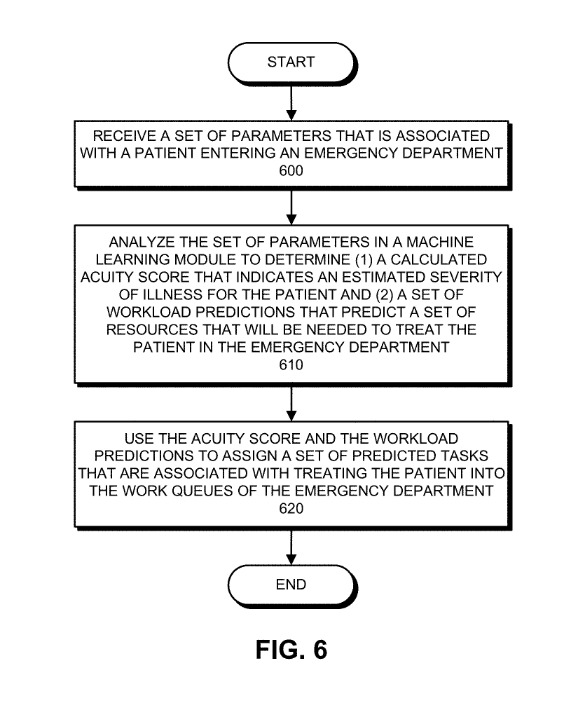

FIG. 6 presents a flow chart that illustrates the process of optimizing emergency department resource management. During operation, a system receives a set of parameters that is associated with a patient entering an emergency department (operation 600). The system analyzes the set of parameters in a machine learning module to determine (1) a calculated acuity score that indicates an estimated severity of illness for the patient and (2) a set of workload predictions that predict a set of resources that will be needed to treat the patient in the emergency department (operation 610). The system then uses the acuity score and the workload predictions to assign a set of predicted tasks that are associated with treating the patient into the work queues of the emergency department (operation 620).

In summary, embodiments of the disclosed techniques associate patient input parameters with acuity, workload predictions, and predicted patient outcomes. Precise acuity assessments and resource prediction can be leveraged to improve patient flows, reducing the length of stays in the ED and the average time to see a provider. Such effects improve patient care and lower overall costs, and improve over time as more input-outcome data is collected and further improve system predictions. Emergency department administration can leverage the tracked data to plan budgets and adjust staff resources in a manner that ensures quality, safety and patient satisfaction while reducing costs.

### Computing Environment

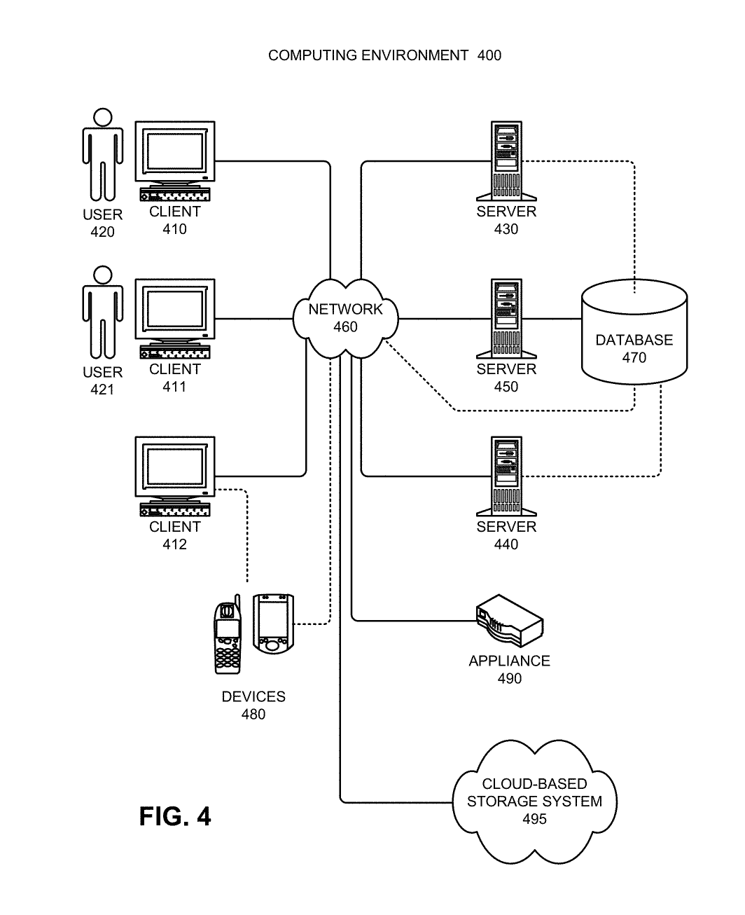

In summary, embodiments of the present invention facilitate optimizing emergency department resource management. In some embodiments of the present invention, techniques for assessing patient issues and predicting emergency department workloads can be incorporated into a wide range of computing devices in a computing environment. For example, FIG. 4 illustrates a computing environment 400 in accordance with an embodiment of the present invention. Computing environment 400 includes a number of computer systems, which can generally include any type of computer system based on a microprocessor, a mainframe computer, a digital signal processor, a portable computing device, a personal organizer, a device controller, or a computational engine within an appliance. More specifically, referring to FIG. 4, computing environment 400 includes clients 410-412, users 420 and 421, servers 430-450, network 460, database 470, devices 480, appliance 490, and cloud-based storage system 495.

Clients 410-412 can include any node on a network that includes computational capability and includes a mechanism for communicating across the network. Additionally, clients 410-412 may comprise a tier in an n-tier application architecture, wherein clients 410-412 perform as servers (servicing requests from lower tiers or users), and wherein clients 410-412 perform as clients (forwarding the requests to a higher tier).

Similarly, servers 430-450 can generally include any node on a network including a mechanism for servicing requests from a client for computational and/or data storage resources. Servers 430-450 can participate in an advanced computing cluster, or can act as stand-alone servers. For instance, computing environment 400 can include a large number of compute nodes that are organized into a computing cluster and/or server farm. In one embodiment of the present invention, server 440 is an online “hot spare” of server 450.

Users 420 and 421 can include: an individual; a group of individuals; an organization; a group of organizations; a computing system; a group of computing systems; or any other entity that can interact with computing environment 400.

Network 460 can include any type of wired or wireless communication channel capable of coupling together computing nodes. This includes, but is not limited to, a local area network, a wide area network, or a combination of networks. In one embodiment of the present invention, network 460 includes the Internet. In some embodiments of the present invention, network 460 includes phone and cellular phone networks.

Database 470 can include any type of system for storing data in non-volatile storage. This includes, but is not limited to, systems based upon magnetic, optical, or magneto-optical storage devices, as well as storage devices based on flash memory and/or battery-backed up memory. Note that database 470 can be coupled: to a server (such as server 450), to a client, or directly to a network. Alternatively, other entities in computing environment 400 (e.g., servers 430-450) may also store such data.

Devices 480 can include any type of electronic device that can be coupled to a client, such as client 412. This includes, but is not limited to, cell phones, personal digital assistants (PDAs), smartphones, personal music players (such as MP3 players), gaming systems, digital cameras, portable storage media, or any other device that can be coupled to the client. Note that, in some embodiments of the present invention, devices 480 can be coupled directly to network 460 and can function in the same manner as clients 410-412.

Appliance 490 can include any type of appliance that can be coupled to network 460. This includes, but is not limited to, routers, switches, load balancers, network accelerators, and specialty processors. Appliance 490 may act as a gateway, a proxy, or a translator between server 440 and network 460.

Cloud-based storage system 495 can include any type of networked storage devices (e.g., a federation of homogeneous or heterogeneous storage devices) that together provide data storage capabilities to one or more servers and/or clients.

Note that different embodiments of the present invention may use different system configurations, and are not limited to the system configuration illustrated in computing environment 400. In general, any device that includes computational and storage capabilities may incorporate elements of the present invention.

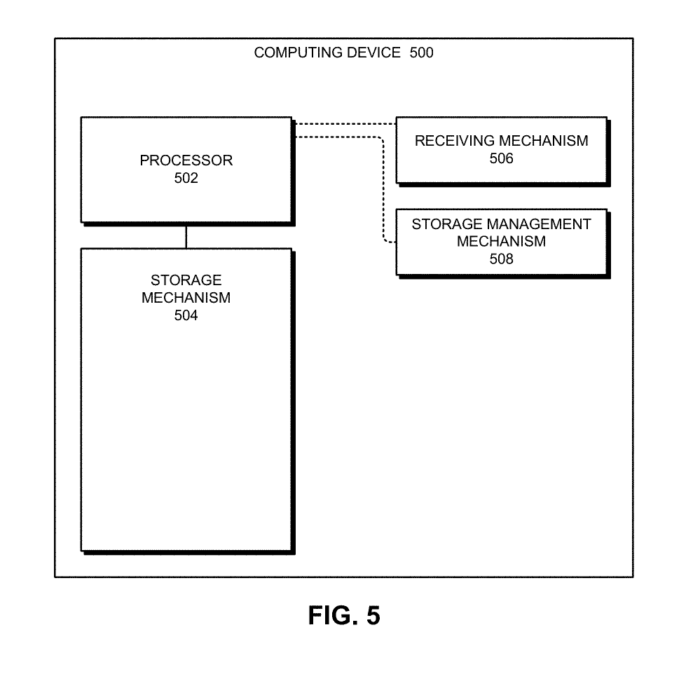

FIG. 5 illustrates a computing device 500 that includes a processor 502 and a storage mechanism 504. Computing device 500 also includes a receiving mechanism 506 and a storage management mechanism 508.

In some embodiments, computing device 500 uses receiving mechanism 506, storage management mechanism 508, and storage mechanism 504 to manage and process emergency department data. For instance, storage mechanism 504 can store emergency department tracking data, and computing device 500 can use receiving mechanism 506 to receive a request to assess a patient and add the patient into the appropriate emergency department queue. Program instructions executing on processor 502 can analyze tracked data to determine workload predictions for a set of patient parameters. Storage management mechanism 508 can manage the databases of information needed for patient assessment and workload predictions, and to access remote electronic health records.

In some embodiments of the present invention, some or all aspects of receiving mechanism 506, storage management mechanism 508, and/or a filesystem device driver can be implemented as dedicated hardware modules in computing device 500. These hardware modules can include, but are not limited to, processor chips, application-specific integrated circuit (ASIC) chips, field-programmable gate arrays (FPGAs), memory chips, and other programmable-logic devices now known or later developed.

Processor 502 can include one or more specialized circuits for performing the operations of the mechanisms. Alternatively, some or all of the operations of receiving mechanism 506 and storage management mechanism 508 may be performed using general-purpose circuits in processor 502 that are configured using processor instructions. Thus, while FIG. 5 illustrates receiving mechanism 506 and/or storage management mechanism 508 as being external to processor 502, in alternative embodiments some or all of these mechanisms can be internal to processor 502.

In these embodiments, when the external hardware modules are activated, the hardware modules perform the methods and processes included within the hardware modules. For example, in some embodiments of the present invention, the hardware module includes one or more dedicated circuits for performing the operations described above. As another example, in some embodiments of the present invention, the hardware module is a general-purpose computational circuit (e.g., a microprocessor or an ASIC), and when the hardware module is activated, the hardware module executes program code (e.g., BIOS, firmware, etc.) that configures the general-purpose circuits to perform the operations described above.

The foregoing descriptions of various embodiments have been presented only for purposes of illustration and description. They are not intended to be exhaustive or to limit the present invention to the forms disclosed. Accordingly, many modifications and variations will be apparent to practitioners skilled in the art. Additionally, the above disclosure is not intended to limit the present invention. The scope of the present invention is defined by the appended claims.

What is claimed is:

1. A computer-implemented method for optimizing emergency department resource management, the method comprising:
receiving a set of parameters that is associated with a patient entering an emergency department;
analyzing the set of parameters in a machine learning module to determine (1) a calculated acuity score that indicates an estimated severity of illness for the patient and (2) a set of workload predictions that predict a set of resources that will be needed to treat the patient in the emergency department; and
using the acuity score and the workload predictions to assign a set of predicted tasks that are associated with treating the patient into the work queues of the emergency department.

2. The computer-implemented method of claim 1, wherein the set of parameters comprises:
a set of patient vital information that is measured at the time the patient checks in to the emergency department, comprising the patient's heart rate, blood oxygen levels, and blood pressure;
a set of patient information recorded by a triage nurse, comprising the patient's age and chief complaint; and
a set of identifying information that is used to access the patient's medical history via an electronic health record for the patient, wherein information from the patient's medical history is included in the set of parameters analyzed by the machine learning module.

3. The computer-implemented method of claim 2,
wherein the set of patient vital information is measured in real-time using a hardware-based palm reader that also reads a set of biometric information from the patient; and
wherein the set of identifying information is compared to the set of read biometric information to authenticate the patient to facilitate access to the patient's electronic health record.

4. The computer-implemented method of claim 2,
wherein the set of information recorded by the triage nurse further comprises an intuited numerical acuity score that is determined by the triage nurse based on the triage nurse's experience, training, and gestalt;
wherein analyzing the set of parameters further comprises comparing the calculated acuity score and the intuited acuity score to check that the triage nurse and the machine learning module approximately agree in the assessment of the patient and are not missing any illness factors; and
wherein if the calculated acuity score and the intuited acuity score diverge substantially the method further comprises flagging a warning and allocating additional resources to the patient to determine an accurate acuity level for the patient.

5. The computer-implemented method of claim 1,
wherein the machine learning module tracks (1) a set of patient and emergency department input parameters for patients who visited the emergency department previously, (2) the workload that was used to treat each patient, and (3) the set of outcomes for each previous patient;
wherein the machine learning module uses supervised machine learning techniques to match the set of parameters for the current patient to a statistically matching set of tracked input parameters to determine from the tracked input parameters the set of workloads predictions and a predicted outcome for the patient.

6. The computer-implemented method of claim 5, wherein the set of tracked parameters used by the machine learning module for supervised machine learning comprises:
an emergency department census at the time of the patient's assignment;
an inpatient census at time of the patient's assignment;
the patient's ambulatory status;
the means of patient arrival;
the patient's triage vital signs and timestamp;
the patient's blood pressure;
the patient's heart rate;
the patient's respiratory rate;
the patient's temperature and method of measurement;
the patient's weight;
the patient's pulse oxygen saturation;
the patient's problem list and medical history;
the patient's current medications;
the patient's allergies;
the patient's social history;
the patient's registration timestamp;
an emergency department provider to which the patient was assigned and a timestamp for the provider assignment;
an emergency department nurse to which the patient was assigned and a timestamp for the nurse assignment;
an emergency department room to which the patient was assigned and a timestamp for the room assignment;
a set of studies ordered for the patient, along with the timestamps of when the studies were ordered, when the studies had changes, and when the studies were completed;
a disposition decision for the patient and a timestamp for the disposition decision;
an assignment for an inpatient bed for the patient and a timestamp for the assignment of the inpatient bed;
a final disposition for the patient and a timestamp for the final disposition;
a billing level of service associated with the patient; and
current environmental conditions for geographic vicinity of the emergency department for the timeframe in which the patient entered the emergency department.

7. The computer-implemented method of claim 1,
wherein determining the calculated acuity score that indicates the estimated severity of illness for the patient involves using the calculated acuity score to sort the patient into one of a set of four acuity groupings; and
wherein the set of four acuity groupings comprises (1) resuscitation, (2) high acuity, (3) medium acuity, and (4) low acuity.

8. The computer-implemented method of claim 1, wherein the set of workload predictions predict:
a set of medications that will need to be administered to the patient;
a set of laboratory orders that will need to be applied for the patient;
a set of radiology orders that will need to taken for the patient;
an amount of time that a physician will need to spend with the patient; and
a set of procedures that the patient will need to undergo.

9. The computer-implemented method of claim 1, wherein assigning the set of predicted tasks further comprises considering:
the shift times for the staff currently working in the emergency department;
current and predicted patient arrival patterns for the emergency department; and
the acuity scores and workload predictions for all of the patients who are currently active in the queues of the emergency department.

10. The computer-implemented method of claim 9, wherein assigning the set of predicted tasks further comprises prioritizing the flow of patients through the radiology, laboratory, and discharge queues.

11. The computer-implemented method of claim 9,
wherein the patient is immediately assigned to a specific physician at the time that the set of predicted tasks are assigned into the work queues of the emergency department; and
wherein immediately assigning the patient to the specific physician provides accountability that leads to better care and efficiency for tasks associated with the patient.

12. The computer-implemented method of claim 1, wherein assigning the predicted tasks into the work queues further comprises sending push notifications to the set of staff and resources associated with a treatment plan for the patient to notify them of their new assignments for the patient.

13. The computer-implemented method of claim 1, wherein the method further comprises determining a crowding score for the emergency department based on:
the number of patients currently being treated by the emergency department;
the acuity scores of the patients currently being treated by the emergency department;
the number of patients currently arriving at the emergency department;
the set of resources available in the emergency department;
the status of the radiology, laboratory, and discharge queues for the emergency department; and
the number of hospital patients currently boarding in the emergency department.

14. The computer-implemented method of claim 13, wherein the method further comprises
using the crowding score to calculate a crowding coefficient for the emergency department; and
adjusting the active set of resources of the emergency department based on the crowding coefficient by performing at least one of:
activating additional staff resources for the emergency department in real-time to reduce a bottleneck; and
deactivating one or more staff resources for the emergency department that are currently underused and predicted to not be used in a proximate timeframe in real-time to reduce costs.

15. The computer-implemented method of claim 14, wherein assigning the set of predicted tasks further comprises determining a physician for the patient based on:
the calculated acuity score;
a workload prediction for the patient that is associated with physician time, effort, and experience;
the physician's shift time;
the length of the physician's shift;
the crowding coefficient; and
the number of patients being treated by the physician and the other physicians who are currently active in the emergency department.

16. The computer-implemented method of claim 1, wherein the method further comprises:
determining that a task associated with the patient is exceeding a predicted workload that was associated with the task;
rebalancing one or more queues that are affected by the excessive task in real-time using the acuity scores and workload predictions of all of the other patients in the emergency department to adjust for the excessive task; and
after the patient has been treated, updating the machine learning module with the actual workloads that were performed for the patient so that the patient's workloads can be used to train the machine learning module in future predictions.

17. A non-transitory computer-readable storage medium storing instructions that when executed by a computer cause the computer to perform a method for optimizing emergency department resource management, the method comprising:
receiving a set of parameters that is associated with a patient entering an emergency department;
analyzing the set of parameters in a machine learning module to determine (1) a calculated acuity score that indicates an estimated severity of illness for the patient and (2) a set of workload predictions that predict a set of resources that will be needed to treat the patient in the emergency department; and
using the acuity score and the workload predictions to assign a set of predicted tasks that are associated with treating the patient into the work queues of the emergency department.

18. A system that optimizes emergency department resource management, comprising:
a processor that supports tracking patient data and performing supervised machine learning techniques to determine acuity scores and workload predictions;
a storage mechanism that stores a patient treatment history for a historical set of patient visits to an emergency department; and
a storage management mechanism;
wherein the processor receives a set of parameters that is associated with a patient entering the emergency department;
wherein the processor is configured to invoke a machine learning module that accesses the storage mechanism via the storage management mechanism to analyze the set of parameters in the context of the stored patient treatment history to determine (1) a calculated acuity score that indicates an estimated severity of illness for the patient and (2) a set of workload predictions that predict a set of resources that will be needed to treat the patient in the emergency department; and
wherein the processor is configured to use the acuity score and the workload predictions to assign a set of predicted tasks that are associated with treating the patient into the work queues of the emergency department.
